(19) United States
(12) Patent Application Publication
(10) Pub. No.: US 2024/0230427 A1
(43) Pub. Date: Jul. 11, 2024

(54) TEMPERATRUE MONITORING SYSTEM

(71) Applicant: Tippmann Engineering, LLC, Fort Wayne, IN (US)

(72) Inventors: Robert T. Tippmann, Fort Wayne, IN (US); Daniel J. Tippmann, Fort Wayne, IN (US); Aaron Mark Engle, Huntertown, IN (US); Kent Eldon Crouse, Huntertown, IN (US)

(21) Appl. No.: 18/407,715

(22) Filed: Jan. 9, 2024

Related U.S. Application Data
(60) Provisional application No. 63/438,148, filed on Jan. 10, 2023.
(60) Provisional application No. 63/462,760, filed on Apr. 28, 2023.

(51) Int. Cl. G01K 13/00; A61L 2/04; A61L 2/24; G01K 1/024

(52) U.S. Cl. CPC G01K 13/00; A61L 2/04; A61L 2/24; G01K 1/024; A61L 2202/14

(57) ABSTRACT

Systems and methods for measuring a temperature of a product are provided. An example method includes providing a temperature probe assembly. The temperature probe assembly includes a temperature probe configured to sense a temperature, a body in electrical communication with the temperature probe, and a scanner fixed to the body. The method further includes scanning a code, via the scanner, and receiving a temperature reading, via the temperature probe. The temperature reading corresponds to a temperature of a product in which the temperature probe is inserted. The method further includes outputting an indication of the temperature reading, based on the code.

### CROSS-REFERENCE TO RELATED APPLICATION

This application claims the benefit of U.S. Provisional Patent Application Ser. No. 63/438,148, filed Jan. 10, 2023 and U.S. Provisional Patent Application Ser. No. 63/462,760 filed Apr. 28, 2023, both entitled TEMPERATURE MONITORING SYSTEM, the entire disclosures of which are hereby expressly incorporated herein by reference.

### BACKGROUND

### 1. Technical Field

The present disclosure relates to temperature monitoring and, in particular, to temperature monitoring systems for palletized goods subject to a temperature change.

### 2. Description of the Related Art

Temperature probes can be used to measure the internal temperature of products. For food products such as meat, monitoring internal temperature may be necessary to ensure safety of the product for later consumption. Internal temperature may be of interest during cooling, warming, or storage, for example, to ensure bacterial growth is prevented or inhibited in accordance with established safety standards.

### SUMMARY

The present disclosure provides a temperature monitoring system including a temperature probe which measures an internal temperature of a product. The measured temperature may be exported to a digital storage medium, and the resulting data may be used for various system analyses and controls.

In some examples, a system for monitoring temperature of a product is provided. The system includes at least one temperature probe assembly; and a docking station that is configured to receive the temperature probe assembly in a docked position. The docking station is configured to create an electrical connection to the temperature probe assembly when the temperature probe assembly is in the docked position, and the docking station is programmed to communicate with the temperature probe assembly in a leader/follower relationship.

In some examples, a temperature probe assembly is provided that includes a temperature probe configured to sense a temperature; and a wireless transmitting module which, when executed by a processor, enables: transmitting a first temperature reading from the temperature probe corresponding to a temperature of a product, to determine whether a target temperature has been reached in the product; and, after the step of transmitting the first temperature, transmitting a second temperature reading from the temperature probe that corresponds to an increase in temperature greater than the target temperature.

In some examples, a method for measuring a temperature of a product is provided. The method includes providing a temperature probe assembly. The temperature probe assembly includes a temperature probe configured to sense a temperature, a body in electrical communication with the temperature probe, and a scanner fixed to the body. The method further includes scanning a code, via the scanner, and receiving a temperature reading, via the temperature probe. The temperature reading corresponds to a temperature of a product in which the temperature probe is inserted. The method further includes outputting an indication of the temperature reading, based on the code.

Depending upon embodiment, one or more benefits may be achieved. These benefits and various additional objects, features and advantages of the present disclosure can be fully appreciated with reference to the detailed description and accompanying drawings that follow.

### BRIEF DESCRIPTION OF THE DRAWINGS

The above mentioned and other features of this invention, and the manner of attaining them, will become more apparent and the invention itself will be better understood by reference to the following description of embodiments of the invention taken in conjunction with the accompanying drawings, where:

FIG. 1 is a top, front, perspective view of a temperature monitoring system made in accordance with the present disclosure;

FIG. 2 is side, rear perspective view of the temperature monitoring system shown in FIG. 1;

FIG. 3 is a top perspective view of a probe assembly of the temperature monitoring system of FIG. 1;

FIG. 4 is another top perspective view of the probe assembly of FIG. 3;

FIG. 5 is a top perspective, exploded view of the probe assembly of FIG. 3;

FIG. 6 is a bottom perspective view of the temperature monitoring system shown in FIG. 1;

FIG. 7 is a perspective view of a controller of the temperature monitoring system shown in FIG. 1;

FIG. 8 is a schematic control system in accordance with the present disclosure;

FIG. 9 is an example method of measuring temperature of a product, according to some aspects provided herein;

FIG. 10 is an example method of measuring temperature of a product, according to some aspects provided herein;

FIG. 11 is an example method of measuring temperature of a product, according to some aspects provided herein; and

FIG. 12 is a schematic device made in accordance with the present disclosure. Corresponding reference characters indicate corresponding parts throughout the several views. Unless stated otherwise the drawings are proportional.

### DETAILED DESCRIPTION

The embodiments disclosed below are not intended to be exhaustive or to limit the invention to the precise forms disclosed in the following detailed description. Rather, the embodiments are chosen and described so that others skilled in the art may utilize their teachings. Aspects of the embodiments may be practiced as methods, systems, or device. Accordingly, aspects of the embodiments may take the form of hardware implementation, an entirely software implementation, or an implementation combining hardware and software.

As mentioned above, temperature probes can be used to measure the internal temperature of products. For food products such as meat, monitoring internal temperature may be necessary to ensure safety of the product for later consumption. Internal temperature may be of interest during cooling, warming, or storage, for example, to ensure bacterial growth is prevented or inhibited in accordance with established safety standards. However, existing temperature probes may be difficult to remove from frozen products, be unable to trace the history of a particular product, and/or allow predictions of when a product will be done freezing. Advantages of aspects disclosed herein may address these deficiencies, while providing additional and/or alternative advantages that will be recognized by those of ordinary skill in the art, at least in light of the present disclosure.

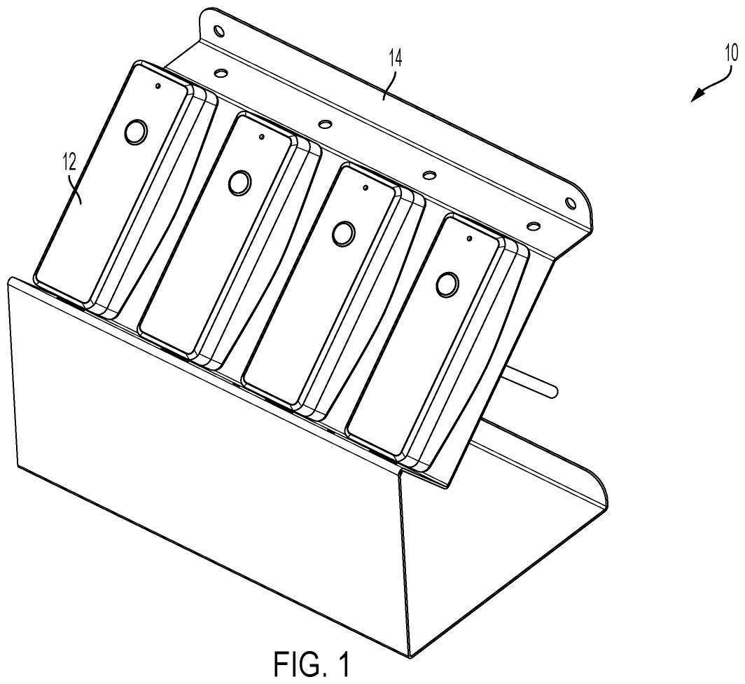

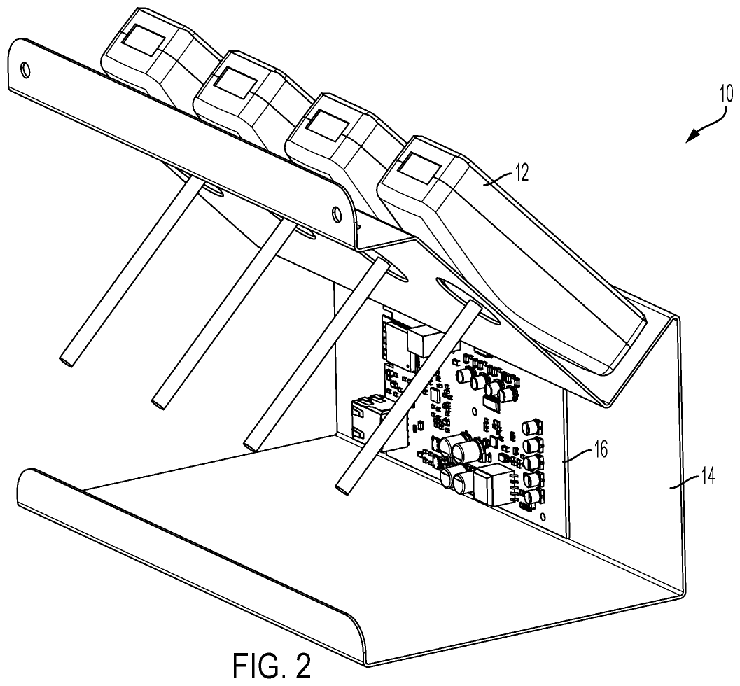

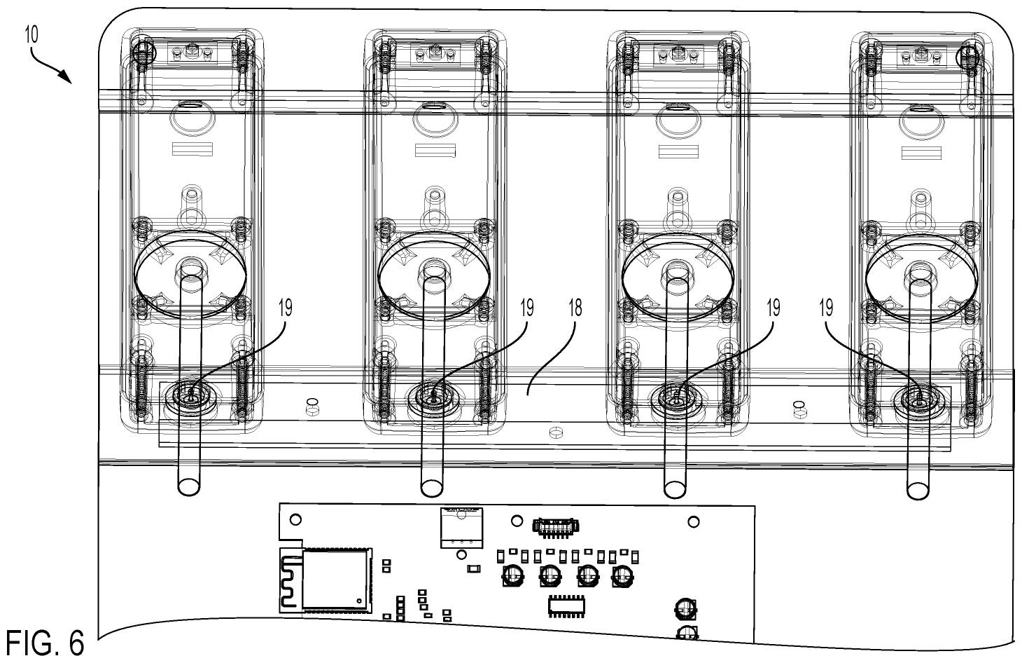

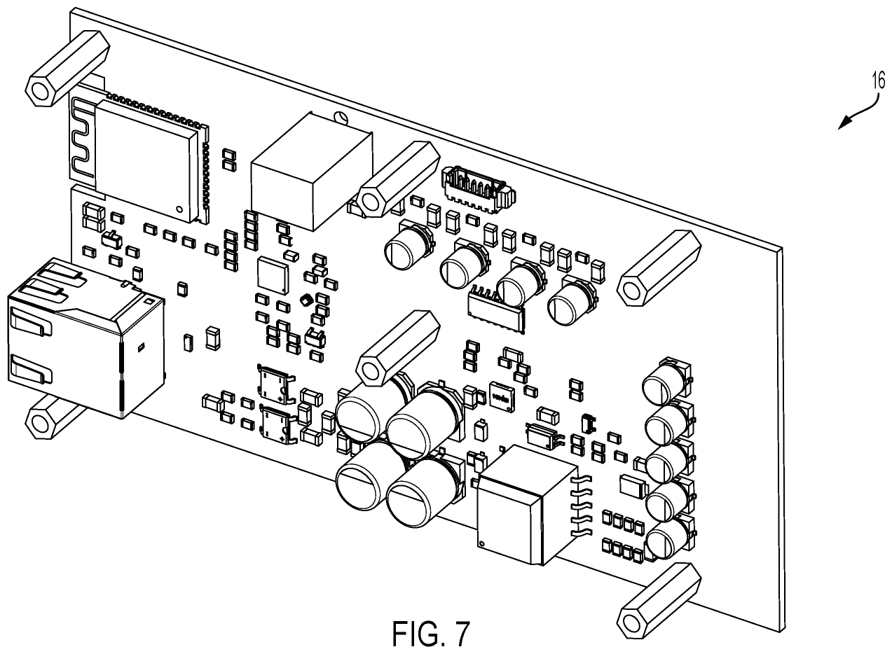

FIG. 1 illustrates a temperature probe system 10 made in accordance with the present disclosure. System 10 includes a plurality of temperature probe assemblies 12, illustratively a set of four probes 12. Each of the probes 12 is received by a docking station 14 having a receiver 18 attached thereto, as shown in FIG. 6. A controller, such as controller 16 shown in FIGS. 2 and 7, communicates with probes 12 via radio frequency (RF) signals.

When probes 12 are docked at docking station 14, docking station 14 receives identity information from probes 12, and also communicates with a remote computer system via RF signals, as described in detail below.

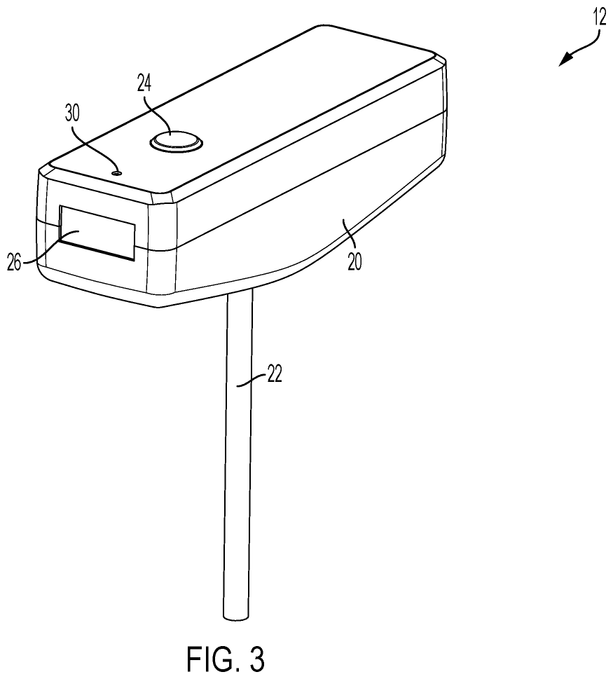

Referring now to FIGS. 3-5, temperature probe assembly 12 includes body 20 with probe 22 extending outwardly therefrom. Probe 22 is configured to be inserted into a product when temperature, and particularly temperature change over time, is desired to be measured. Probe 22 mayinclude an elongate straight shank with a sharpened tip to facilitate insertion into a product to be measured. The probe 22 maycooperate with the body 20 to measure a temperature of the product at a given moment in time (e.g., in Celsius, Fahrenheit, etc.). Additionally, or alternatively, the probe 22 and body 20 maymeasure a change in temperature over a specified period of time (e.g., 1 millisecond, 1 second, 1 minute, 1 hour, etc.), such that a rate of change of the temperature of the product with respect to the specified period of time may be determined.

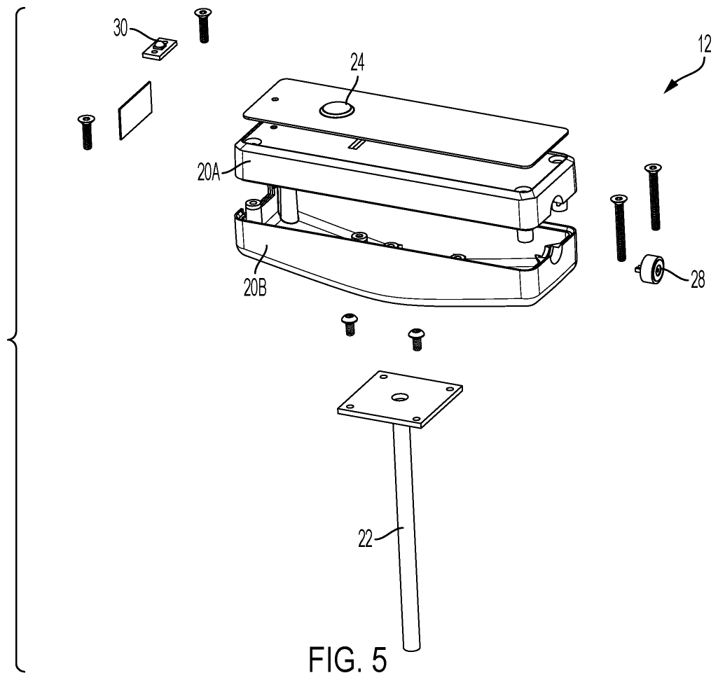

In the illustrative embodiment of FIGS. 3-5, body 20 is fixed to probe 22. For example, the elongate shank of the probe 22 may be fixed (e.g., welded) to a base plate (FIG. 5) which can be removably or permanently coupled to body 20. In alternative embodiments, body 20 may be separate from probe 22, and connected thereto by an electrical wire. Referring to FIG. 5, body 20 mayinclude upper portion 20A and lower portion 20B, which may be selectively fixed to one another to enclose an interior cavity housing a battery, an internal printed circuit board (PCB), and other electronics as described herein.

As described further below, temperature probe 12 is designed to log the measured temperature of the target product internally, creating a data record which also cross references with additional information to create a complete record that can be used for superior cooling system diagnostics and control.

Temperature probe assembly 12 includes a scanner 26, shown in FIG. 3. Scanner 26 is configured to scan a product code, such as a barcode, quick response (QR) code, fiducial marker, or the like. Scanner 26 may be used to scan a product code affixed to a product case or box, and the product code may have embedded information including the type and quantity of product contained in the case, temporal information such as packing date and current date, warehouse location, and other information. Probe assembly 12 can record the information embedded in the product code and link this information to the subsequently (or previously) gathered time and temperature information gathered via probe 22.

Scanner 26 mayalso be used to gather other information pertinent to a pallet/warehouse management system. For example, a pallet upon which a given case it stored may have its own product code, sometimes referred to as a “license plate” for the pallet. This information may also be scanned by scanner 26 and linked with the case to which temperature probe 12 is applied. An employee-handler of the case, who may be using the probe 12 to gather temperature data, may also have a unique identifier, such as a barcode or QR code. The employee may scan his or her own identity code to be stored with the product information of the cases under management.

System 10 mayalso include an air temperature sensor (not shown) which measures and records the ambient air temperature near probes 12 and produces a time-stamped temperature record of the same. The resulting data stream may be temporally linked with the data stored by one or more probe assemblies 12 for use by controller 16, or another controller, as described herein.

Probe assembly 12 mayinclude a heating device extending along probe 22 and electrically powered by a battery contained within body 20. For example, a wire may extend along the probe 22 which can be heated by electrical resistance upon activation. Activation may occur by a user action, such as by interfacing with a user-interface (e.g., pushing button 24 of FIG. 3, providing an audio command, touch-screen command, gesture command, key command, or the like). Upon activation, the probe 22 is heated causing the local area around the probe 22 to warm. This heating may be effected by a resistive element embedded within the elongate portion of probe 22 which receives electrical current from an electrical source, such as a battery contained within body 20, for example. When probe 22 is embedded in a frozen product, such as a frozen meat product, this heating may be used to locally soften the product in the immediate vicinity of the probe 22, allowing for easy extraction from otherwise frozen material without a significant volume of warming or thawing of the frozen product itself. For example, less than 1% of a volume of a case of frozen product may be warmed more than 10 degrees Fahrenheit by activation of the heating function of probe 22.

In some embodiments, the heating of probe 22 mayfacilitate extraction from a frozen meat product in under a minute after activation of button 24. Accordingly, initiating the heating device of the probe 22 mayenable extraction of the probe 22 from the product (e.g., otherwise the probe 22 may be relatively stuck in the product until the product unfreezes enough for the probe to be extracted).

In some examples, a freeze test may be performed on a product that tests how long it takes for a product to reach a target temperature. A freeze test may start at a desired or configurable amount of time after the probe assembly 12 is removed from the docking station 14 to allow for time to insert the probe assembly 12 into a product. For example, the configurable amount of time may be about 15 minutes. The freeze test may finish when a target temperature of the product is reached, at which time the probe assembly 12 may generate a summary of the freeze test. In some examples, data included in the summary may correspond to time and/or temperature of the product, including a total time to reach the target temperature from the beginning of the freeze cycle. In some examples, the summary of the freeze test is only a single line of data. The summary may be uploaded into a data store, such as a spreadsheet. This upload may occur upon generation, e.g., the probe assembly 12 may transmit the summary to a computing device and/or server (described below) at its next periodic data transfer. Alternatively, this upload can be accomplished at other times such as when the probe assembly 12 is docked at the docking station 14. The data store may include a plurality of summaries that each correspond to a respective freeze test from a plurality of previously-performed freeze tests. For example, the data store may include an all-time history of freeze cycles accomplished for any particular one probe assembly 12.

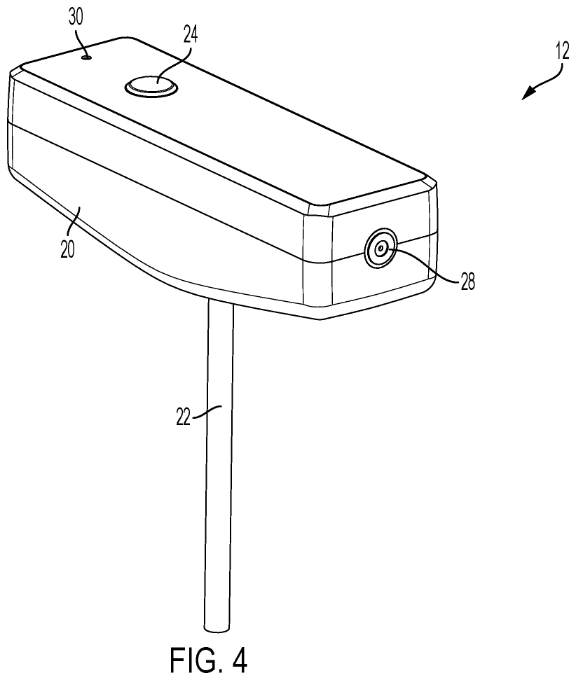

Turning again to FIGS. 1 and 2, probe assemblies 12 are shown in a seated position on docking station 14. Docking station 14 may be connected to a source of external power, such as via a power over ethernet (POE) connection which also serves as a data transfer connection. Alternatively, power may be received via standard 120VAC or 240VAC building power. When a probe assembly 12 is docked, its electrical port 28 (FIG. 4) makes electrical contact with a corresponding electrical port 19 of docking station 14 (FIG. 6). In the illustrative embodiment of FIG. 6, docking station 14 mayinclude receiver 18, which includes a series of electrical ports 19 positioned and configured to engage the corresponding plurality of probe assemblies 12 of system 10. When a probe assembly 12 is docked in a slot of the docking station 14, the resulting electrical contact made between the corresponding electrical port 19 and the probe assembly 12 mayinclude two sources of electrical flow: charging and data transmission.

For charging, an internal battery of probe assembly 12, which may be contained within body 20, may receive a flow of electrical power via electrical connectors 18, 19 when probe assembly 12 is docked. The battery, when fully charged, may be sized to continuously operate probe assembly 12 for more than one day, such as at least a week, without connection to the external power source. This allows probe assembly 12 to collect data, as further described herein, for at least as long as a temperature-change procedure (e.g., freezing, cooling, thawing, or warming) may take to accomplish.

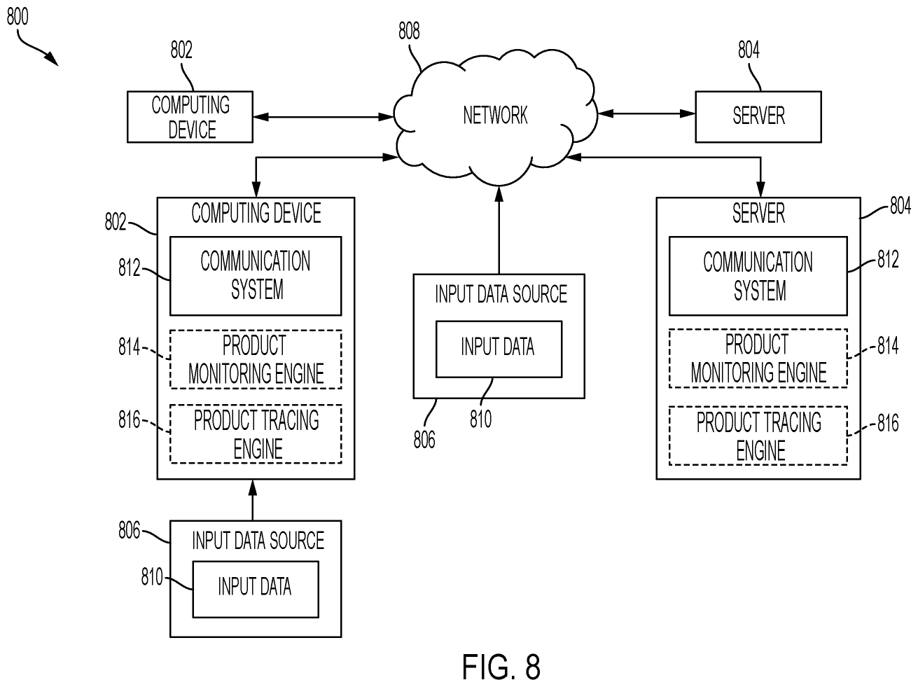

For data transmission, the electrical connection made between port 19 and connector 18 mayprompt data logging and storage within temperature probe assembly 12 to transfer data received from probe assemblies 12 to an external and/or centralized computing system, referred to herein as an “external/centralized system.” This external/centralized system may include data storage and processing, such as a central server, a cloud server, or the like, as shown in FIG. 8 and further described below. In particular, this “hard” or physical electrical connection between port 19 and connector 18 may be in addition to a wireless connection used by probe assembly 12, and may be used to identify an individual probe assembly 12 and prompt data transmission to and from the probe. For example, to the extent that the wireless connection from the probe assembly 12 mayhave been incomplete or imperfect prior to connection with the docking station 14, docking probe assembly 12 at docking station 14 mayprompt probe assembly 12 to transmit any missing or corrupted data packets. In some examples, the external/centralized system may include a single server or computing device. In some examples, the external/centralized system may include a plurality of servers or computing devices, such as in a distributed computing system.

Additionally, docking station 14 may be connected (such by an ethernet cable and/or wireless connections) to the internet and, thereby, to the external/centralized system.

In an embodiment, docking station 14 may be considered a “leader” or “master” while one or more probes 12 may be considered “followers” or “slaves.” That is, docking station 14 may issue a prompt to the controller to retrieve and impart native programming to probes 12 regardless of prior programming which may be stored in probe 12. Probe assembly 12 may be reprogrammed with this native programming each time it is docked on docking station 14.

In this way, docking station 14 may be used to create associations with any given probe assembly 12 and a larger system with which the docking station 14 is associated. For example, controller 16 of docking station 14 may be programmed with an association for a specific user/customer owning system 10, and this association may also be uploaded to the external/centralized system. When any given probe assembly 12 is docked on that docking station 14, controller 16 mayinduce the external/centralized system to “take ownership” of that probe assembly 12, creating associations between the probe assembly 12 and the user/customer that owns docking station 14. This allows new or otherwise unassociated probe assemblies 12 to be integrated into system 10 simply and quickly by the owner of system 10, allowing for easy expansion of system 10 or replacement of existing probes 12. In some examples, instead of mediating communications through the external/centralized system, the docking station 14 and the probe 12 maycommunicate directly with one another.

Controller 16 of docking station 14 may be programmed with identifying information for the organization that owns or controls the docking station 14. For example, a name, Employer Identification Number (EIN) or other identity signifier of a company or other consortium may be programmed into controller 16 and thereby associated with docking station 14. This identity signifier may then be uploaded to the external/centralized system and automatically programmed into probes 12 upon being placed in their docked positions (FIG. 1). This ensures that probes 12 used in connection with an operation associated with docking station 14 are property identified as such, without any manual reprogramming or other user intervention required except for seating the probes 12 into the docking station 14, a process which is done in any case for recharging purposes.

While owner identity is a suitable link with docking station 14 for many purposes, other identities may be used as required or desired for a particular application. For example, controller 16 mayinstead be programmed with a particular user identity such that a specific one of a team of personnel is associated with docking station 14, and thereby with any probes 12 that are docked with that docking station 14.

The above-described leader/follower modality for docking station 14 and probes 12, respectively, facilitates modularity of system 10. In particular, probes 12 may be replaced, added or moved freely and associated or re-associated with a particular operation by simply docking probes 12 in that operation's docking station 14. This facilitates warranty repairs, for example, by allowing probes 12 to be replaced or exchanged without any prior programming specific to the operation where the probes will be used.

Similarly, probes 12 can be freely exchanged between operational sites and reprogrammed for their next use at the new site by simply docking the probes 12 at the new site (i.e., in its own proprietary docking station 14). For an operation using online or centralized tools to monitor the status of their probes 12, as described further below, docking a new or transferred probe 12 into the operation's docking station 14 mayalso automatically add the probe 12 to the centralized monitoring tool. For example, the probe 12 may be associated with a first network setting (e.g., of a first warehouse), then the probe 12 may be put into the docking station 14 (e.g., associated with a second warehouse) and the probe 12 may be updated to be associated with a second network setting (e.g., of the second warehouse). Additional and/or alternative settings or telemetry data discussed herein may be updated based on the probe 12 being put into the docking station 14, after being previously associated with another docking station (e.g., which may be similar in aspects to the docking station 14).

As noted above, probe assembly 12 mayalso include a wireless transmitting module capable of wireless transmitting data periodically or in real time to the external/centralized system. In some embodiments, collection of telemetry data (further described below) by probe 12 mayoccur at an interval on the order of seconds to minutes, while the transmission of the resulting collected telemetry data may be wireless transmitted from the probe 12 to a central server less frequently than the collection interval, such as at an interval on the order of minutes to hours. In one embodiment, telemetry data may be collected about every 2-10 minutes, while transmission of the collected data to the external/centralized system may occur about every 45-90 minutes. The server external/centralized system may be on-site at the operation linked to the docking station 14, or may be offsite, such as in a cloud-based server.

Probe assembly 12 contains and collects various data, including settings and telemetry data. Settings are data pertaining the function and status of probe 12, while telemetry data are data collected by probe 12 during operation.

Telemetry data include: target temperature; product temperature as measured by probe 22, such as the temperature of product contained within a case; ambient or air temperature as measured by body 20, such as the temperature around the case of product; probe sensor serial number associated with probe assembly 12; battery temperature for the internal battery of probe assembly 12; battery voltage for the internal battery of probe assembly 12; state of charge for the internal battery of probe assembly 12; probe state or status; and all barcodes that were scanned by scanner 26, such as a rack identification, a freezer system identification (e.g., a modular air handler as described in U.S. patent application Ser. No. 16/938,837, incorporated by reference below), stock keeping unit(s) (SKUs) for products, pallet product code or “license plate,” messaging notification recipient information such as phone numbers for SMS text messaging and email notification addresses, and any other uncategorized barcodes which may have been scanned by scanner 26.

Settings data include: service set identifier (SSID) such as network name and password information for local WiFi connectivity; how often temp is measured (i.e., the sample interval); how often to connect to the server to report data (i.e., the transmission interval) for both docked and non-docked status of the probe assembly 12; the identity of servers to which data is sent; and identities of specific employees to whom one or more indications associated with the telemetry data are sent (e.g., to a computing device associated with the specific employee).

An online or internet-based dashboard may be provided to view and manage data collected and transmitted by the probe(s) 12 of system 10. The dashboard may contain one or more indications corresponding to all, or a subset, of the telemetry data discussed herein. The dashboard may be generated by a computing device and/or server device (e.g., computing device 802 and/or server 804 discussed with respect to FIG. 8). The dashboard may include a graphical user-interface (GUI) that includes indications corresponding to one or more aspects of the telemetry data collected by probe 12. The dashboard may include derivative calculations, forecasts, and/or predictions based, at least in part, on the telemetry data collected by probe 12 (e.g., an indication of when a product in which the probe 12 is located is forecasted to reach a target temperature). The GUI may be displayed on a display screen of a computing device (e.g., the computing device 802). A first GUI may be generated based on a first set of telemetry data that is received and a second GUI may be updated based on a second set of telemetry data that is received. The second GUI may replace the first GUI on the display screen.

The system 10 may cooperate with the external/centralized system and, as needed, controller 16, to send messages to cellular phones, tablets or other computers when a temperature-change process (e.g., a freezing of cased product) is complete. The messages may be sent to a single device and/or a plurality of devices. The messages may be sent to devices based on which employee scanned their own identity code to be stored with the product information of the cases under management.

In some examples, users may scan their own codes (e.g., bar codes, QR codes, or other fiduciary markers), using the scanner 26, based on employee shifts for a warehouse.

Therefore, one or more employees associated with a current shift may receive the messages discussed above. Additionally, or alternatively, one or more employees who are not associated with a current shift may receive the messages based on an organizational hierarchy (e.g., a manager, supervisor, or executive may receive a message whether or not they are associated with the current shift). In some examples, mechanisms disclosed herein may receive a first indication corresponding to a first code being scanned. The first code may be associated with a first user (e.g., an employee on a first shift). Accordingly, a message may be sent to a first device (e.g., associated with the first user), based on the first code being scanned. Subsequently, mechanisms described herein may receive a second indication corresponding to a second code being scanned. The second code may be associated with a second user (e.g., an employee on a second shift). Accordingly, a message may be sent to a second device (e.g., associated with the second user), based on the second code being scanned.

System 10 may be used in connection with blast freezer applications, in which palletized arrangements of cases of product are frozen for later storage and transport. Examples of such systems, and other systems compatible with system 10, may be found in U.S. patent application Ser. No. 16/938,837 filed Jul. 24, 2020 and entitled MODULAR HEAT TRANSFER SYSTEM (Attorney Docket: TPE0023-02-US), which is co-owned with the present application, the entire disclosure of which is hereby incorporated herein by reference.

Signals and data collected to probe(s) 12 and transmitted to an external/centralized system or other controller may be used to automatically control equipment associated with a temperature-change operation. For example, with probe 12 inserted into a product to be frozen from a non-frozen state, time-stamped temperature data may be used to assess whether a target temperature has been reached in the product. When the target temperature reaches a threshold, such as −10 degrees C., fans inducing cold-air flows around the product may be shut off to conserve energy. Control over other systems, including chillers, alerts, alarms and any other warehouse equipment, may also be effected by system 10 in connection with data collected and transmitted by probe(s) 12.

In examples where probe 12 is inserted into a product to be frozen from a non-frozen state, the probe 12 may be removed (e.g., automatically and/or manually) from the product in response to determining that the target temperature has been reached in the product. An indication corresponding to an increase in temperature may be received (e.g., from the probe 12) by one or more devices disclosed herein in response to determining that the target temperature has been reached in the product, such as because the probe 12 has been removed from the product that is being frozen and/or because equipment for freezing the product has been shut off.

For example, probe assembly 12 may be programmed with a “beacon mode” in which probe assembly 12 issues signals to the central server indicating a need for an operator to locate and attend to the probe assembly 12. Beacon mode may be activated, for example, when the temperature data received by probe assembly 12 indicates that the temperature-change process is complete. Beacon mode may be activated, as another example, when the probe loses connection from a server and/or computing device. Similarly, probe assembly 12 may be placed into a “lost mode” by a communication or signal from the external/centralized system. In the lost mode, the probe assembly 12 issues a signal to aid in tracing the location of probe assembly 12. In some examples, when the probe assembly 12 is in beacon mode or lost mode, a visual and/or audio indication may be provided on or near the probe 12 to assist in locating the probe assembly 12. For example, the system 10 (and/or the probe 12) may include a light 30, such as a light emitting diode (LED), that illuminates when the probe 12 is in beacon mode or lost mode. Additionally, or alternatively, the system 10 (and/or the probe 12) may include a speaker that emits audio when the probe assembly 12 is in beacon mode or lost mode.

Probe assembly 12 may be further programmed with an “auto sanitize mode” in which a heater associated with probe 22 is activated while probe assembly 12 is seated in docking station 14 or otherwise not placed in a product for temperature measurement. For example, when probe assembly 12 is placed in the docking station 12, auto sanitize mode activates the probe heater and monitors the temperature of the probe 22 until it reaches a setpoint between 80° C. and 100° C. When this setpoint temperature is achieved, the probe heater remains activated to hold the setpoint temperature for a designated time, such as about 3-5 minutes, after which the probe heater is deactivated and auto sanitize mode is considered completed. Auto sanitize mode acts to kill any residual bacteria that might be present on the probe 22, such as bacteria left from previously-frozen meat products. Advantageously, auto sanitize mode provides a safeguard against insufficient or ineffective manual cleaning procedures, such as when a worker fails to sterilize the probe 22 using an alcohol wipe or other appropriate sterilization products.

System 10 allows for live, real-time traceability of products as they are subject to temperature-change processes. Additionally, the data collected during the temperature-change processes may be used for validation of the process specifics. For example, meat products may be subjected to testing and procedures to prevent handling procedures which could lead to a risk for trichinosis. The data collected by system 10 can be used to demonstrate proper handling procedures and adherence to safety standards, such as may be required for quality-control evaluation and auditing.

When probe 12 is docked and makes a connection between electrical ports 28 and 19 and described above, probe 12 mayinitiate a charging protocol. The charging protocol includes accepting a flow of current from port 19 to the battery housed by body 20.

Additionally, probe 12 mayreconfigure its wireless connection (e.g., with local WiFi as described above) and/or initiate a different data connection protocol with the external/centralized system (e.g., by increasing the sampling rate for data transmission). If connection to the docking station 14 fails but charging of the battery continues, probe 12 may be programmed to continue attempts to connect to the docking station 14 as long as it remains docked, rather than reverting back to a non-connected configuration. On the other hand, when probe 12 is not connected at port 28 to the docking station 14, probe 12 is programmed to create a wireless connection in accordance with its in-operation, rather than docked, configuration.

FIG. 8 shows an example of a system 800 including an external/centralized system as described above in accordance with some aspects of the disclosed subject matter.

The system 800 may be a system for monitoring temperatures of palletized goods, including system 10 or a similar configuration. The system 800 includes one or more computing devices 802, one or more servers 804, one or more input data sources 806, and a communication network or network 808. In some examples, the computing device 802 can receive input data 810 from the input data source 806. Additionally, or alternatively, in some examples, the network 808 can receive input data 810 from the input data source 806.

In some examples, computing device 802 includes a communication system 812, product monitoring engine or component 814, and/or a product tracing engine or component 816. In some embodiments, computing device 802 can execute at least a portion of the product monitoring engine 814 to receive temperature data corresponding to a product based on the input data 810. In some examples, the computing device 802 can execute at least a portion of the product tracing engine 816 to determine a user (e.g., employee) to whom a notification regarding the product should be sent. In some examples, the communication system 812 includes a wireless transmitting module which, when executed by a processor, enables transmissions relating to one or more temperature readings.

In some examples, server 804 includes a communication system 812, product monitoring engine or component 814, and/or a product tracing engine or component 816. In some embodiments, server 804 can execute at least a portion of the product monitoring engine 814 to receive temperature data corresponding to a product based on the input data 810. In some examples, the server 804 can execute at least a portion of the product tracing engine 816 to determine a user (e.g., employee) to whom a notification regarding the product should be sent. In some examples, the communication system 812 includes a wireless transmitting module which, when executed by a processor, enables transmissions relating to one or more temperature readings or other telemetry or settings data as described above.

Additionally, or alternatively, in some examples, computing device 802 can communicate data received from input data source 806 to the server 804 over a communication network 808, which can execute at least a portion of the product monitoring component 814 and/or the product tracing component 816. In some examples, the product monitoring component 814 executes one or more portions of methods/processes disclosed herein and/or recognized by those of ordinary skill in the art, in light of the present disclosure. In some examples, the product tracing component 816 executes one or more portions of methods/processes disclosed herein and/or recognized by those of ordinary skill in the art, in light of the present disclosure.

In some examples, computing device 802 and/or server 804 can be any suitable computing device or combination of devices, such as a controller, desktop computer, a vehicle computer, a mobile computing device (e.g., a laptop computer, a smartphone, a tablet computer, a wearable computer, etc.), a server computer, a virtual machine being executed by a physical computing device, a web server, etc. Further, in some examples, there may be a plurality of computing devices 802 and/or a plurality of servers 804.

In some examples, input data source 806 can be any suitable source of input data (e.g., data generated from a computing device, data stored in a repository, data generated from a software application, data received from a temperature probe, data received from a sensor, etc.). In some examples, input data source 806 can include memory storing input data (e.g., local memory of computing device 802, local memory of server 804, cloud storage, portable memory connected to computing device 802, portable memory connected to server 804, etc.). In some examples, input data source 806 can include an application configured to generate input data and provide the input data via a software interface. In some examples, input data source 806 can be local to computing device 802. In some examples, input data source 806 can be remote from computing device 802, and can communicate input data 810 to computing device 802 (and/or server 804) via a communication network (e.g., communication network 808). Input data source 806 can include sources of telemetry data and/or settings data as described herein. In some examples, the input data source 806 may include multiple sources of input data, such as a temperature probe (e.g., probe 12), a cooling system, a heating system, a warehouse management system, etc.

In some examples, the input data 810 may include telemetry data and/or setting data as defined above, as well the identity of servers (e.g., one or more of servers 804) to which data is and/or state information (e.g., on/off or intermediary power levels) for a light, a speaker, a heating system, a cooling system or other devices associated with system 10. Additional and/or alternative attributes of the input data 810 may be recognized by those of ordinary skill in the art at least in light of teachings provided herein.

In some examples, communication network 808 can be any suitable communication network or combination of communication networks. For example, communication network 808 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard) which may include a narrowband Internet of things (NB-IoT) communications protcol, a wired network, etc. In some examples, communication network 808 can be a local area network (LAN), interfaces conforming known communications standard, such as Bluetooth® standard, IEEE 802 standards (e.g., IEEE 802.11), a ZigBee® or similar specification, such as those based on the IEEE 802.15.4 standard, a wide area network (WAN), a public network (e.g., the Internet), a private or semi-private network (e.g., a corporate or university intranet), any other suitable type of network, or any suitable combination of networks. In some examples, communication links (arrows) shown in FIG. 8 can each be any suitable communications link or combination of communication links, such as wired links, fiber optics links, Wi-Fi links, Bluetooth® links, cellular links, satellite links, etc. Communication network 808 may also utilize long range radio communications, referred to as “LoRa” and associated communication protocol and system architecture referred to as “LoRaWAN.” Advantageously, LoRa can provide “Internet of things” type functionality but does not rely on a cellular networks, with a range on the order of tens of kilometers.

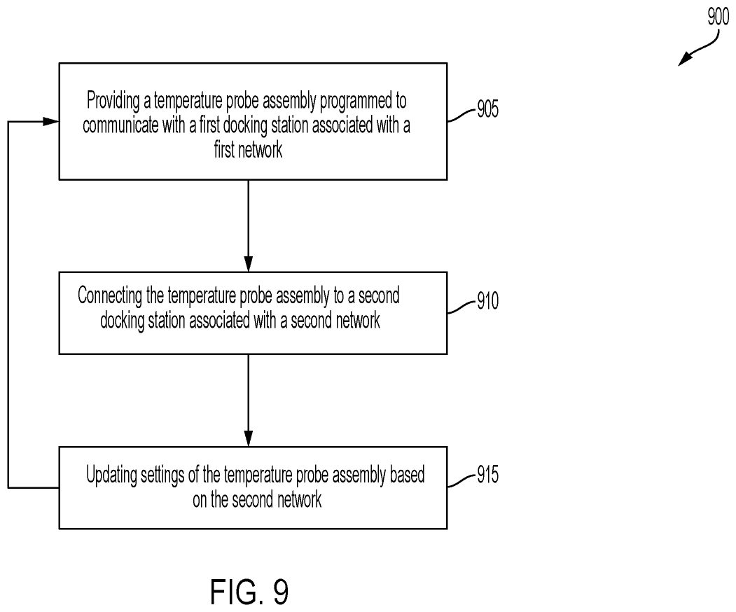

FIG. 9 illustrates an example method 900 of measuring a temperature of a product, according to some aspects described herein. In example, aspects of method 900 are performed by a device, such as computing device 802 and/or server 804, discussed above with respect to FIG. 8.

Method 900 begins at operation 905, wherein a temperature probe assembly is provided. The temperature probe assembly may be similar or identical to the temperature probe assembly 12 discussed earlier herein with respect to FIGS. 1-7. For example, the temperature probe assembly may include a temperature probe that is configured to sense a temperature of a product. The temperature probe assembly may be programmed to associate and communicate with a first docking station (e.g., docking station 14) associated with a first network (e.g., network 808). As described herein, the first docking station may communicate with the temperature probe via an external/centralized system.

At operation 910, the temperature probe assembly may be connected to a second docking station that is associated with a second network. The second docking station may be different than the first docking station and the second network may be different than the first network.

Generally, the temperature probe assembly may be connected to the second docking station when the temperature probe assembly is transported from a first warehouse to a second warehouse, and/or from a distributor to a client, and/or from a first location with a warehouse to a second location within the warehouse.

At operation 915, one or more settings of the temperature probe assembly may be updated based on the second network. The updated settings may include setting data as described herein, such as one or more from the group of: updating a sampling interval of the temperature probe assembly, updating a target temperature reading, updating an indication of a pallet location, and/or updating a specific employee to whom one or more indications associated with the telemetry data are sent. Additional and/or alternative settings of the temperature probe assembly which may be updated will be recognized by those of ordinary skill in the art.

Accordingly, the temperature probe assembly may be updated from settings that are specific to a first location or installation (e.g., a first warehouse, a distributor/client, a first location within a warehouse, etc.) to setting that are specific to a second location or installation (e.g., a second warehouse, a client/distributor, a second location within the warehouse, etc.).

Method 900 may terminate at operation 915. Alternatively, method 900 may return to operation 905 to provide an iterative loop of updating settings of a temperature probe assembly depending on to which docking station of a plurality of docking stations the temperature probe assembly was last connected.

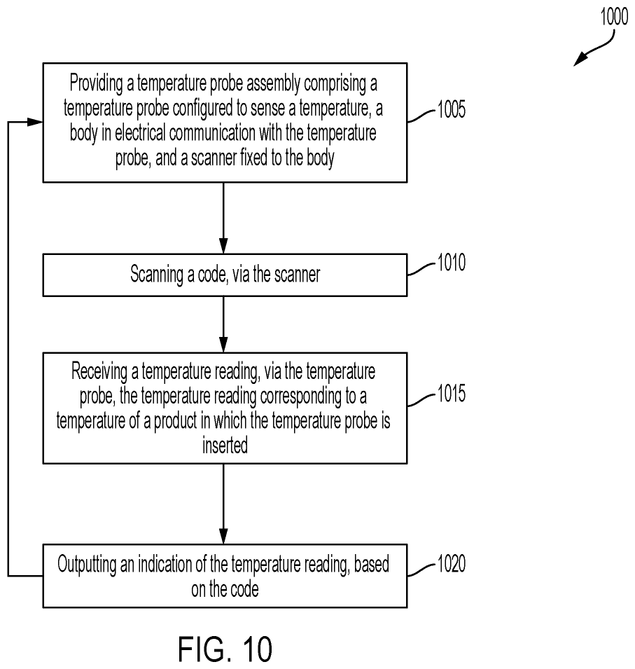

FIG. 10 illustrates an example method 1000 of measuring a temperature of a product, according to some aspects described herein. In example, aspects of method 1000 are performed by a device, such as computing device 802 and/or server 804, discussed above with respect to FIG. 8.

Method 1000 begins at operation 1005, wherein a temperature probe assembly is provided, such as temperature probe 12 described herein. The temperature probe assembly includes a temperature probe configured to sense a temperature (e.g., probe 22), a body in electrical communication with the temperature probe (e.g., body 20), and a scanner fixed to the body (e.g., scanner 26). The temperature probe assembly may be similar or identical to the temperature probe assembly 12 discussed earlier herein with respect to FIGS. 1-7.

At operation 1010, a code is scanned, via the scanner. In some examples, the code is a product code associated with a product in which the temperature probe is inserted.

In some examples, the code corresponds to a user. In some examples, the code includes a bar code, a QR code, or another fiduciary marker that may be recognized by those of ordinary skill in the art.

At operation 1015, a temperature reading is received, via the temperature probe. The temperature reading corresponds to a temperature of a product in which the temperature probe is inserted. In some examples, the temperature probe assembly includes a heating device that extends along the temperature probe, and the heating device may be initiated (e.g., generate heat) to enable extraction of the temperature probe from the product. Specifically, in some examples, the temperature probe may be collecting readings from a product that is frozen. Accordingly, to remove the temperature probe from the frozen product, it may be helpful to generate heat that softens at least a localized portion of the product surrounding the temperature probe, such that the temperature probe may be extracted without significant thawing or warming of the frozen product.

At operation 1020, an indication of the temperature reading is output, based on the code. For example, the indication may be output as an audio message, and/or as a visual indication, and/or as a message transmitted to a component of a local device and/or remote device. Additional and/or alternative examples of the manner and form in which the indication of the temperature reading is output will be recognized by those of ordinary skill in the art.

In some examples, the indication associates the product (in which the temperature probe is inserted) with the temperature reading. In some examples, the indication is sent to a device associated with the user to whom the code corresponds. In some examples, the code scanned at operation 1010 is a first code that corresponds to a first user, and the device to which the indication is sent is a first device. The method 1000 maythen further include scanning a second code that corresponds to a second user, via the scanner, and sending to indication to a second device associated with the second user.

In some examples, the temperature probe assembly collects telemetry data as described herein. As noted above, telemetry data may include target temperature, product temperature, ambient temperature, probe sensor serial number, battery temperature, battery voltage, state of charge, probe state or status, and scanned barcodes.

In some examples, the method 1000 mayinclude calculating when the product in which the temperature probe is located is forecasted to reach a target temperature. In such examples, the indication output at operation 1020 mayinclude the calculated forecast. The forecast may be determined based on the received temperature reading, based on a plurality of temperature readings including the received temperature reading, and/or based on the specific product in which the temperature probe is inserted.

Method 1000 mayterminate at operation 1020. Alternatively, method 1000 may return to operation 1005 to provide an iterative loop of providing a temperature probe assembly to collect temperature readings and output indications thereof based on scanned codes.

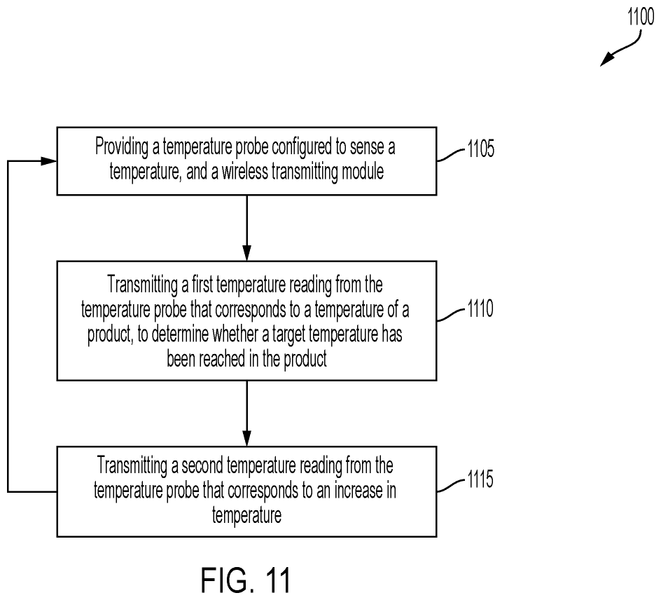

FIG. 11 illustrates an example method 1100 of measuring a temperature of a product, according to some aspects described herein. In example, aspects of method 1100 are performed by a device, such as computing device 802 and/or server 804, discussed above with respect to FIG. 8.

Method 1100 begins at operation 1105, wherein a temperature probe and a wireless transmitting module are provided. The temperature probe is configured to sense a temperature. The temperature probe may be similar or identical to the temperature probe 12 discussed earlier herein with respect to FIGS. 1-7.

In some examples, the temperature probe is part of a temperature probe assembly that includes a body (e.g., body 20) in electrical communication with the temperature probe (e.g., probe 22), a battery contained within the body, and a heating device extending along the probe. The heating device may be powered by the battery and enable the increase in temperature. In some examples, the temperature probe assembly further include a scanner (e.g., scanner 26) fixed to the body.

In some examples, the temperature probe assembly associated with method 1100 collects telemetry data as described herein.

At operation 1110, a first temperature reading is transmitted from the temperature probe that corresponds to a temperature of a product. The transmission of the first temperature reading enables a determination as to whether a target temperature has been reached in the product. For example, the transmission may occur from the wireless transmitting module, and the determination as to whether the target temperature has been reached may be performed on a remote computing device (e.g., server 804), based on and in response to the transmission of the first temperature reading.

At operation 1115, a second temperature reading is transmitted from the temperature probe that corresponds to an increase in temperature. For example, the temperature may increase due to the temperature probe being removed from a product in which it was located. In some examples, the temperature increases due to the heating device being enabled to allow for ease of extraction of the temperature probe. In some examples, the temperature increases due to cooling equipment (e.g., fans) being turned off, when the cooling equipment is directed at the product in which the temperature probe is located, and the target temperature of the product has been reached.

In some examples, the method 1100 further includes activating a beacon mode in response to determining that the target temperature has been reached in the product. The activating a beacon mode may include providing an indication to locate the temperature probe. For example, the temperature probe assembly may include a speaker and the indication may be an audio indication emitted from the speaker. Additionally, or alternatively, the temperature probe assembly may include a light, such as a light emitting diode (LED), and the providing an indication may include illuminating the light (e.g., LED).

Method 1100 mayterminate at operation 1115. Alternatively, method 1100 may return to operation 1105 to provide an iterative loop of transmitting a first temperature reading to determine whether a target temperature has been reached in a product, and transmitting a second temperature reading that corresponds to an increase in temperature (e.g., as a result of determining that the target temperature has been reached in the product).

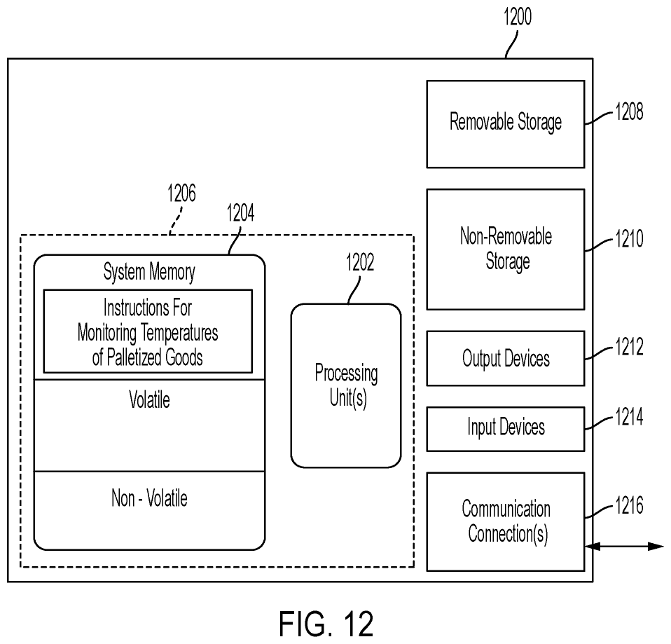

FIG. 12 illustrates a simplified block diagram of a device with which aspects of the present disclosure may be practiced in accordance with aspects of the present disclosure. The device may be a mobile computing device, for example. One or more of the present embodiments may be implemented in an operating environment 1200. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smartphones, network PCs, minicomputers, mainframe computers, distributed computing environments that include any of the above systems or devices, and the like.

In its most basic configuration, the operating environment 1200 typically includes at least one processing unit 1202 and memory 1204. Depending on the exact configuration and type of computing device, memory 1204 (e.g., instructions for one or more aspects disclosed herein, such as one or more aspects of methods/processes discussed herein) may be volatile (such as RAM), non-volatile (such as ROM, flash memory, etc.), or some combination of the two. This most basic configuration is illustrated in FIG. 12 by dashed line 1206. Further, the operating environment 1200 mayalso include storage devices (removable, 1208, and/or non-removable, 1210) including, but not limited to, magnetic or optical disks or tape, flash drives, solid-state drives (SSD), and the like. Similarly, the operating environment 1200 mayalso have input device(s) 1214 such as remote controller, keyboard, mouse, pen, voice input, on-board sensors, etc. and/or output device(s) 1212 such as a display, speakers, printer, motors, etc. Also included in the environment may be one or more communication connections 1216, such as LAN, WAN, a near-field communications network, a cellular broadband network, point to point, etc.

Operating environment 1200 typically includes at least some form of computer readable media. Computer readable media can be any available media that can be accessed by the at least one processing unit 1202 or other devices comprising the operating environment.

By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, flash drives, solid-state drives (SSD), or any other tangible, non-transitory medium which can be used to store the desired information. Computer storage media does not include communication media. Computer storage media does not include a carrier wave or other propagated or modulated data signal.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media.

The operating environment 1200 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A system for monitoring temperature of a product, comprising:
at least one temperature probe assembly; and
a docking station configured to selectively receive the temperature probe assembly in a docked position, the docking station configured to create an electrical connection to the temperature probe assembly when the temperature probe assembly is in the docked position, and the docking station programmed to communicate with the temperature probe assembly in a leader/follower relationship.

2. The system of claim 1, wherein the docking station is associated with a first network, and wherein the temperature probe assembly has settings associated with the first network.

3. The system of claim 1, wherein the docking station is a first docking station, wherein the system further comprises a second docking station associated with a second network, and wherein the temperature probe assembly is configured to update its settings based on the second network when connected to the second docking station.

4. The system of claim 1, wherein the temperature probe assembly further comprises:
a temperature probe configured to sense a temperature of the product;
a body in electrical communication with the temperature probe;
a battery disposed within the body;
an electrical connector fixed to the body, the electrical connector configured to charge the battery; and
a communication device disposed within the body.

5. The system of claim 4, wherein the temperature probe assembly further comprises a scanner fixed to the body.

6. The system of claim 1, wherein telemetry data collected by the temperature probe assembly includes a target temperature, a product temperature as measured by the temperature probe, and an ambient temperature as measured by the temperature probe.

7. The system of claim 6, wherein the telemetry data further includes a probe sensor serial number, a temperature of a battery of the temperature probe, a voltage of the battery, a state of charge of the battery, a probe state or status, and at least one scanned barcode.

8. A temperature probe assembly comprising:
a temperature probe configured to sense a temperature; and
a wireless transmitting module which, when executed by a processor, enables:
transmitting a first temperature reading from the temperature probe corresponding to a temperature of a product, to determine whether a target temperature has been reached in the product; and
after the step of transmitting the first temperature, transmitting a second temperature reading from the temperature probe corresponding to an increase in temperature greater than the target temperature.

9. The temperature probe assembly of claim 8, further comprising:
a body in electrical communication with the temperature probe;
a battery contained within the body; and
a heating device extending along the temperature probe, the heating device being powered by the battery and enabling the increase in temperature.

10. The temperature probe assembly of claim 8, wherein the probe is programmed with an auto sanitize mode in which the heating device is activated when the probe assembly is not placed in the product, the heating device remaining activated until a measured temperature of the probe reaches a setpoint temperature between 80° C. and 100° C., the heating device deactivated after the setpoint temperature is held designated time between about 3-5 minutes.

11. The temperature probe assembly of claim 8, wherein the probe is programmed with a beacon mode activated in response to determining that the target temperature has been reached in the product, wherein the beacon mode activates an indication configured to aid in locating the temperature probe.

12. The temperature probe assembly of claim 11, wherein the temperature probe includes a light emitting diode (LED), and wherein the beacon mode includes illuminating the LED.

13. The temperature probe assembly of claim 8, wherein telemetry data collected by the temperature probe assembly includes a target temperature, a product temperature as measured by the temperature probe, and an ambient temperature as measured by the temperature probe.

14. A method for measuring a temperature of a product with a temperature probe assembly, the temperature probe assembly comprising a temperature probe configured to sense a temperature, a body in electrical communication with the temperature probe, and a scanner fixed to the body, the method comprising:
scanning a code, via the scanner;
receiving a temperature reading, via the temperature probe, the temperature reading corresponding to a temperature of a product in which the temperature probe is inserted; and
outputting an indication of the temperature reading, based on the code.

15. The method of claim 14, wherein the code is a product code associated with the product in which the temperature probe is inserted, and wherein the indication associates the product with the temperature reading.

16. The method of claim 14, wherein the code corresponds to a user, and wherein the indication is sent to a device associated with the user.

17. The method of claim 16, wherein the code is a first code, wherein the user is a first user, wherein the device is a first device, wherein the method further comprises scanning a second code corresponding to a second user, via the scanner, and wherein the indication is sent to a second device associated with the second user.

18. The method of claim 14, wherein the temperature probe assembly further comprises a heating device extending along the temperature probe, and wherein initiating the heating device enables extraction of the temperature probe from the product.

19. The method of claim 14, further comprising collecting telemetry data via the temperature probe assembly, the telemetry data comprising a target temperature, a product temperature as measured by the temperature probe, and an ambient temperature as measured by the temperature probe.

20. The method of claim 14, further comprising:
calculating when the product in which the temperature probe is located is forecasted to reach a target temperature,
wherein the indication comprises the calculated forecast.
